(19) United States
(12) Patent Application Publication
(10) Pub. No.: US 2015/0066523 A1
(43) Pub. Date: Mar. 5, 2015

(54) TELEMEDICINE INFORMATION SYSTEM

(71) Applicant: EBM TECHNOLOGIES INCORPORATED, TAIPEI CITY (TW)

(72) Inventor: WILLIAM PAN, TAIPEI CITY (TW)

(73) Assignee: EBM TECHNOLOGIES INCORPORATED, TAIPEI CITY (TW)

(21) Appl. No.: 14/016,210

(22) Filed: Sep. 2, 2013

(51) Int. Cl. G06F 19/00; A61B 5/00

(52) U.S. Cl. CPC G06F 19/3418; A61B 5/0022
USPC 705/2

(57) ABSTRACT

Disclosure herein is related to a telemedicine information system. The telemedicine information system includes a monitoring end which receives the physiological signals generated by one or more physiological sensors disposed at the client end over a network. The medical staff uses monitoring terminal device at the monitoring end. The monitoring terminal device is connected with a monitoring server for acquiring the physiological signals since the server periodically retrieves and records the signals generated by the client end's sensors. The signals will be converted to the meaningful information allowing the medical staff to monitor the remote client's physiological condition. The claimed system achieves an efficient remote medical process since the person at the monitoring end may anytime acknowledge the client's condition.

### BACKGROUND OF THE INVENTION

1. Field of the Invention

The present invention is generally related to a telemedicine information system; more particularly to a monitoring end disposed with a device for anytime obtaining physiological condition of the client end, and timely issuing a warning signal; the device at monitoring end and a monitoring server form the telemedicine information system.

2. Description of Related Art

With the enhancement of network bandwidth and well development of network technology, the various related applications are widespread to the general users. For example, in order to provide health care to the users, the network technology may be applied to use of telemedicine at a distance. That means the telemedicine provides the home users at home the health care at a distance, including the any medical aid from the remote medical staff.

The kind of telemedicine technology may incorporate the modern computer network technologies. The telemedicine may be associated with the computer, communication, and audio/video processing technologies. The telemedicine provides the medical care at a distance, and broader range of medical service made by the medical institutions since it combines the well-developed network technology and the medical profession. In particular, the service is directed to the disabled persons, or the persons at regions that are inaccessible and lack of medical facilities.

The general telemedicine service is such as voice/video call for remote interrogation, remote care, television monitoring, or even the doctor's diagnosis made according to physiological data retrieved from the client end's monitoring equipment.

### SUMMARY OF THE INVENTION

Disclosure is related to a telemedicine information system according to the present invention. This system renders an effective telemedicine operation through the present network.

The telemedicine information system of the present invention is associated with one or more physiological sensing devices disposed at the user end. The physiological sensing device is used to sense the physiological condition of the user. The system disposes a monitoring server used to acquire the physiological signals at the monitoring end according to one embodiment of the present invention. The monitoring server is connected with the one or more physiological sensing devices over a network, and able to periodically retrieve physiological signals generated by the devices at the user end. The monitoring server may dispose a subscriber database recording physiological signals and the associated user data. The privilege of accessing the signals in the server is configurable.

The medical staff may hold a monitoring terminal device, for example the tablet computer, to link the monitoring server over the network. The monitoring terminal device may therefore retrieve physiological signals. It is noted that a monitoring program may be installed into the monitoring terminal device for converting the signals into acknowledgeable messages. The messages corresponding to the signals are referred to determine the physiological condition.

The monitoring terminal device includes a data processing unit used to process the physiological signals as the monitoring information. The device also includes a communication unit used to establish a network connection to the monitoring server. Still further, the device includes a display module used to display the monitoring information. A display module is included in the monitoring terminal device for displaying the monitoring information. A memory unit in the monitoring terminal device is used to buffer the physiological signals retrieved from the monitoring server; and to convert the signals to the information to be displayed. Further, an input module is included in the device for rendering an input interface.

When any abnormal condition is found by the monitoring terminal device held by the medical staff, the communication circuit disposed in the monitoring terminal device is initiated to establish a communication channel with the client end device. The communication circuit is such as a video-audio processing circuit used to perform an audio or video call. The user at the client end may use a mobile communication device to receive the messages from the monitoring end. The content made between the medical staff and the user at the client end may be such as the medical recommendation including the hospital according to the client end's location. The positioning function made by the user's handheld mobile communication device is referred to the location-based suggestion made by the medical staff at the monitoring end.

According to one further embodiment of the present invention, a notification technology via a message server may be used to conduct the communication between the monitoring end and the client end.

### BRIEF DESCRIPTION OF THE DRAWINGS

The foregoing aspects and many of the attendant advantages of this invention will be more readily appreciated as the same becomes better understood by reference to the following detailed description, when taken in conjunction with the accompanying drawings, wherein:

FIG. 1 schematically shows a client and a monitoring end in the telemedicine information system according to one embodiment of the present invention;

FIG. 2 shows a schematic diagram illustrating facilities at a monitoring end of the telemedicine information system according to one embodiment of the present invention;

FIG. 3 shows a schematic diagram illustrating the telemedicine information system including a client end and a monitoring end according to one embodiment of the present invention;

FIG. 4 shows a functional diagram illustrating the telemedicine information system according to one embodiment of the present invention;

FIG. 5 schematically shows facilities at a client end and a monitoring end of the telemedicine information system according to one embodiment of the present invention;

FIG. 6 shows a schematic diagram depicting an information interface of a monitoring terminal device;

FIG. 7 schematically shows a chart depicting monitoring information at a monitoring terminal device;

FIG. 8 schematically shows an interface depicting user information at a monitoring terminal device.

### DETAILED DESCRIPTION OF THE PREFERRED EMBODIMENTS

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

At least one of the objectives of the telemedicine information system of the present invention is to provide an efficient, instant, and convenient telemedicine environment. The telemedicine information system according to the disclosure renders a solution that allows medical staff to use a handheld device to remotely monitor patient's physiological chart and messages. Especially, the physiological data is made by a physiological monitor at a client end. Through this remote monitoring scheme, the patient's physiological condition at the client end can be instantly acknowledged by the monitoring end. Therefore, the system timely facilitates medical assistance while any emergency occurs with instant warning signal.

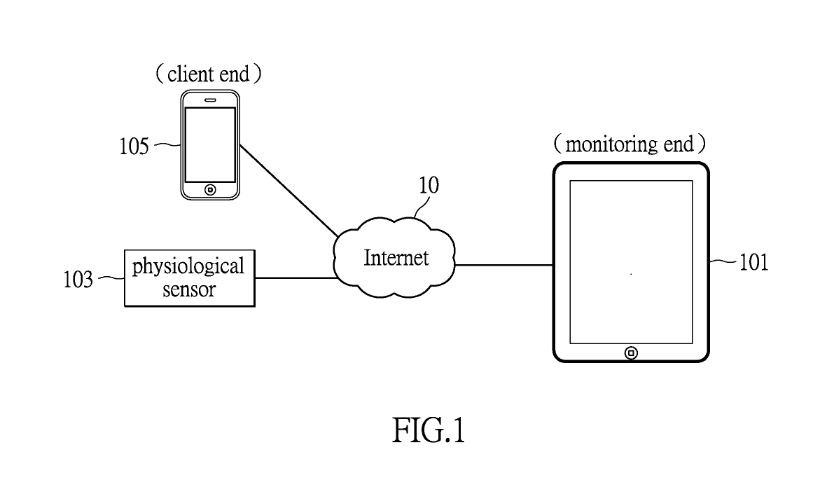

The exemplary framework of the telemedicine information system in accordance with the present invention is referred to FIG. 1. In this schematic diagram, a relationship between a client and a monitoring end of the telemedicine information system is illustrated.

The monitoring end is connected with at least one client end over a network such as Internet 10. The monitoring end may anytime recognize physiological data retrieved from multiple client ends.

In an exemplary example, the medical staff at the monitoring end is such as doctor or/and nurse who is equipped with at least one monitoring terminal device 101. Preferably, the monitoring terminal device 101 is such as a handheld tablet, smart phone, or any computer device that has a display and capability of communication. The physiological data at the client end may be retrieved by a monitoring program operated in the monitoring terminal device 101. The program is able to make diagnosis of the physiological and judge if any abnormal condition according to a judgment rule.

In the figure, a physiological sensor 103 appeared to sense physiological condition for the person to be monitored is installed at the client end. For example, the physiological sensor 103 is such as a heart rhythm sensor, a sphygmometer, oximeter, respiratory frequency measurement instrument, or thermometer. One or in combination of more than one sensor may be installed as required. In general, these physiological instruments have no capability of communication, or with limited functions. However, the telemedicine information system requires the physiological sensors able to link to network, or conduct the communication with plug-in functionality. Alternatively, a communication set 105 at the client end may be configured to make the communication.

If the monitoring end is located remotely according one of the embodiments, the client end may connect with the monitoring end over Internet 10, so as to receive the various signals of physiological sensors. The staff at the monitoring end may monitor the person at the client end through the monitoring terminal device 101. Further, the device 101 allows the staff at the monitoring end to communicate with the person at the client end.

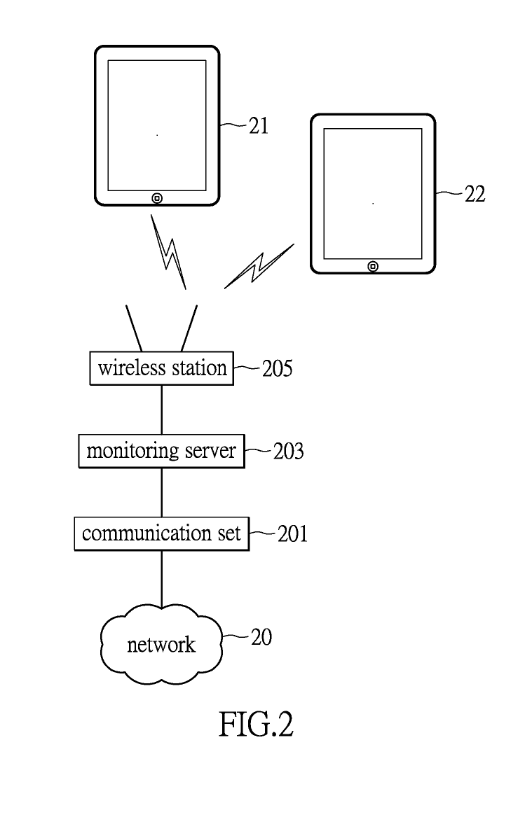

Reference is made to FIG. 2, which depicts facilities at the monitoring end of the telemedicine information system in accordance with the present invention.

A monitoring server 203 is implemented by a computer system. This server 203 includes a database used to record the physiological data retrieved from the client end over a network 20. The database records the various physiological data corresponding to the different physiological sensors/facilities including their hardware information, the patient's basic data, and his pathological data. The pathological data is such as the medical history, doctor's diagnosis record of the person or the patient. At the monitoring end, over the network 20, a communication set 201 serves as the device to bridge the network 20. Medical staff at the monitoring end or the operators may hold the monitoring terminal devices 21, 22 to connect with the monitoring server 203 over a local wireless station 205. Via the monitoring server 203, the staff may be permitted to access the client information and the corresponding physiological data from the database. Furthermore, via the monitoring server 203, the instantaneous physiological data from the client end may also be retrieved.

In an exemplary embodiment, the shown monitoring terminal devices 21, 22 are such as handheld computer device in which programs are installed. The programs preferably include a remote monitoring program adapted to the present invention. The communication circuits are driven to establish connection to the monitoring server 203 when the operating system of the monitoring terminal device 21 or 22 is operated to execute the monitoring program. Based on identification of the operator, or a privilege, the data of client end may be accessed. The client data may include ID and physiological data of each client. The monitoring program may provide function to automatically determine if there is any abnormal condition from the physiological data. The various indicative methods such as flashing, pictorial, vibration, and the like diagrammatic form are used to notice the medical staff.

Still further, the monitoring terminal device 21, 22 allows the medical staff to communicate with the monitored person at the client end. The communication may be conducted by voice call or video call in order to render a location-based medical advice.

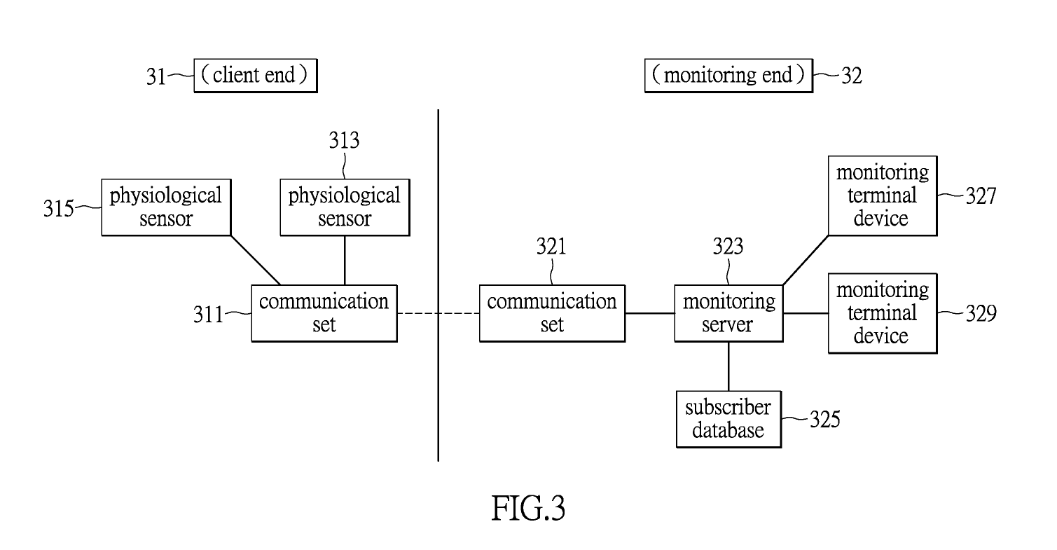

FIG. 3 depicts a connection relationship between the client and the monitoring end of the telemedicine information system according to one embodiment of the invention.

The system essentially includes a client end 31 and a monitoring end 32. At the client end 31, a communication set 311 conducting the communication and its connected physiological sensors 313, 315 are included. The communication set 311 may be a mobile communication device such as mobile phone besides the general network equipment such as the network router. The communication set 311 is used to transmit the signals made by the physiological sensors 313, 315, and the related message may be delivered or displayed onto the other monitoring terminal devices. Further, the signals associated with the physiological sensors 313, 315 may also be integrated for delivery. The example described in the figure may not substantially limit the invention. Alternatively, the physiological sensors 313, 315 may transmit the messages to the monitoring end 32 by their built-in communication modules.

At the monitoring end 32, the monitoring server 323 is configured to collect messages sent from the every client end 31. Further, this monitoring server may also be a platform serving communication means between the staff at the monitoring end 32 and the client end 31. The monitoring server is such as a platform for delivering any message made by video, audio or/and text. The shown monitoring server 323 is communicated with the facilities at the client end 31 via the communication set 321 at the monitoring end 32. More, an encrypted network channel may be established there-between in consideration of information security.

At the monitoring end 32, a subscriber database 325 is configured to record the data for the client end. The subscriber database 325 may include a variety of physiological data for the client end. The monitoring terminal devices 327, 329 are utilized to access the user information in the database 325 within the local network.

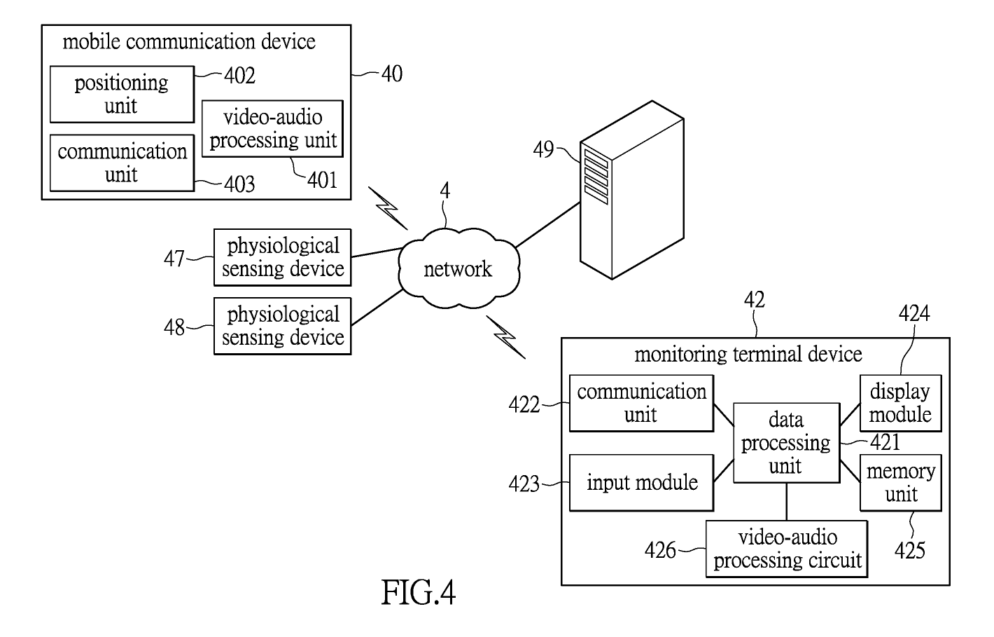

Reference is made to FIG. 4 showing the circuits performing the functions made by the telemedicine information system according to one embodiment of the present invention.

The diagram schematically depicts the functional blocks made by the client end and the monitoring end. In addition to the circuit-based functional modules, some portions of the module may be embodied by software or firmware. In an exemplary embodiment, a monitoring server 49 may be installed at the monitoring end. The monitoring server 49 is such as a computer server that is installed within a LAN. A network 4 (first network connection) is connected made by its inside communication function. Oppositely, one or more physiological sensing devices 47, 48 link to the network 4 by the inside communication circuits or via other additional network device such as the communication device 311 shown in FIG. 3. The physiological sensing devices 47, 48 are connected with the monitoring server 49 may be exemplarily by a proprietary line or a network-based security channel. It is noted that the security channel can ensure the information security and privacy; the mentioned network 4 is preferably, but not limited to, Internet.

The monitoring server 49 is allowed to receive physiological signals from the various physiological sensing devices 47, 48 at every client end periodically. The signals may be stored in the subscriber database (not shown in this figure) based on the every subscriber's identification. The subscriber database stores the physiological signals and the associated user data. The data in the database is provided for the medical staff at the monitor end to authenticate the client's ID and to enquire. The every input made by the medical staff may correspond to one or more clients to be monitored.

At the monitoring end, the medical staff or the other related personnel holds the portable device such as the shown monitoring terminal device 42, for example the general smart phone, tablet computer, laptop computer or the like personal device. In practice, the device may also be shared with some or all of the medical staff for connecting with the monitoring server 49. In one embodiment, the monitoring terminal device 42 is such as a tablet computer by which the medical staff may operate a monitoring program executed in the device 42. A user interface initiated by the monitoring program is used to guide the user to perform authentication, message acquirement, and monitoring physiological signals.

The monitoring server 49, over the first network, connects with the at least one physiological sensing device at the client end for acquiring the physiological signals. While the monitoring server receives the request made by the medical staff who uses the monitoring terminal device 42 for accessing the messages, the server may permit the staff's access request for the client's data according to his privilege. In which, the monitoring program converts the physiological signals to the understandable monitoring messages. The user interface initiated by the monitoring program therefore displays the monitoring messages. The messages may be made to describe any abnormal condition, for example using video, pictures, or lighting. The monitoring program not only allows the staff to access the physiological signals stored in the monitoring server 49, but also to acquire the latest signals instantly for real-time monitoring.

Further, the monitoring terminal device 42 and the monitoring server 49 may be within the same LAN; or alternatively located in different network domains. Over the mentioned second network, the monitoring terminal device 42 is connected to the monitoring server 49. The monitoring terminal device 42 is such as a computer system having a data process unit 421 that is used to process the physiological signals obtained from the monitoring server 49 to be the monitoring information. The data processing unit 421 is a core circuit of the monitoring terminal device 42 in charge of data processing including the signals useful to the present invention. The circuit units in the device 42 are electrically connected with this data processing unit 421. The circuits are such as communication unit 422, input module 423, display module 424, memory unit 425 and video-audio processing circuit 426.

The communication unit 422 is used to establish the second network for the monitoring terminal device 42 to link the monitoring server 49. In an exemplary embodiment, WiFi™ technology constitutes a wireless network to embody the second network that allows the monitoring terminal device 42 to connect with the monitoring server 49. The device 42 may also connect with any remote device over this wireless network. In one embodiment, the monitoring terminal device 42 also transmits message to the device at the client end over the network. The monitoring terminal device 42 may include a display that is exemplarily a touch-sensitive display allowing the user to touch in. The display module 424 is used to display out the image signals, especially the mentioned monitoring information.

The monitoring terminal device 42 includes a memory unit 425 which is such as the built-in or external storage associated to the device 42. The memory unit 425 may be a buffer to buffer the physiological signals and the monitoring information made by the monitoring server 49. The input module 423 includes the inside circuit for processing input signals, and an input interface such as the touch-sensitive display, tangible keyboard, computer mouse, or a gesture sensor.

In addition to the physiological sensing devices 47, 48 at the client end, the user at the client end may also use a mobile communication device 40 such as mobile phone, smart phone, or tablet computer. In particular, the mobile communication device 40 is provided for the user to transmit the physiological signals when the user puts on the portable physiological sensing device(s). Through this mobile communication device 40, the user is allowed to communicate with the medical staff at the monitoring end.

The mobile communication device 40 has built-in communication function, for example the communication unit 403 is activated to establish connection to the network 4. A mobile network or WiFi™ renders the connection directed to the network 4. Over the network 4, the medical staff at the monitoring end is allowed to communicate with the end user by vocal or video call. The mobile communication device 40 may include a video-audio processing unit 401 that is used to process the audio/video signals there-between. In one embodiment, a notification technological may be adopted to conduct the message transmission. For example, the monitoring terminal device 42 transmits messages to the mobile communication device 40 at the client end using a notification message.

In one further embodiment, the mobile communication device 42 may be disposed with a positioning unit 402 for acquiring position thereof. The positioning unit 402 is such as the circuit components of a Global Positioning System (GPS). The positioning unit 42 acquires the device's positioning information that is transmitted to the monitoring end. When the monitoring staff obtains the client's position, any location-based assistance may be provided if any emergency is met. For example, the nearby hospital may be recommended based on the positioning information.

According one of the embodiments of the present invention, a monitoring program is initiated to establish connection with the monitoring server when the medical staff uses the monitoring terminal device to access the client's physiological signals. Then the monitoring server generates a request of ID verification for confirming the staff's privilege to use the monitoring terminal device. The verified ID is allowed to access the subscriber's data. In the meantime, it determines the clients to be monitored when the privilege associated with the medical staff's ID is verified. The medical staff may be permitted to access multiple clients' data and as well to monitor multiple clients.

The client's data may include the each client's ID and the associated physiological signals. The physiological signals include signals instantly obtained from the client end. The signals may also be the monitoring data collected within a period of time for the purpose of diagnosis. The signals may be the accessible history. Those data may be stored and updated in or link to the subscriber database of the monitoring server. Furthermore, the medical staff may modify the diagnosis or give annotation to the content.

It is noted that the monitoring program installed in the monitoring terminal device may be configured to determine the abnormal signals from the physiological signals according to a preset warning threshold. In addition to creating records, the monitoring program is activated to transmit message to the client end. The mentioned notification is one of the methods to conduct the messaging. The medical staff may also therefore decide the method to communicate with the user at the client end. At last, the content may be updated to the database when the medical staff makes diagnosis. It is noted that the process of updating database never ends until all the physiological data are completely checked.

In one further embodiment, when the person to be monitored under the telemedicine information system is a patient who is bedridden in bed, he or she may wear the wearable physiological sensor for a period of time; the physiological sensor may periodically send the physiological data as the sensor is always on line. The monitoring end therefore periodically receives the message from the client end. Any medical decision can be made if any abnormal condition is checked.

In one further embodiment, the person to be monitored may wear the physiological sensor at the moment he/she feels unwell since the physiological sensor is not always put on. Therefore, the physiological data may be generated and sent to the monitoring end at the moment the person put on the sensor. The person to be monitored may activate a communication with the medical staff as he requires. The medical staff at the monitoring end may clearly check the medical data associated with the retrieved physiological data through his/her handheld monitoring terminal device. The staff may provide location-based medical advice to the person, or/and establish a communication channel to the client end. The advice is such as teaching any person at the client end to handle the emergency, or to take medicine. In an exemplary example, multiple medical staffs may simultaneously in consultation over a voice call or video call as a conference call. A suitable nearby hospital may be advised when the monitoring end can position the person to be monitored.

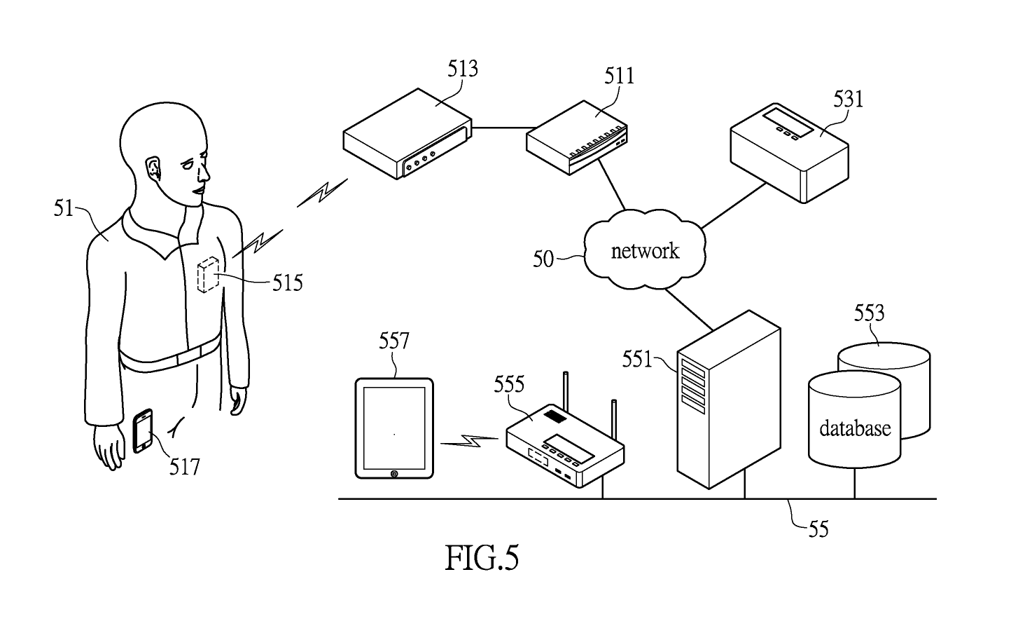

Reference is next made to FIG. 5 schematically depicting the relationship between the client end and the monitoring end in the telemedicine information system.

According to the figure, a to-be-monitored person 51 who wears a physiological sensor is at the client end. The physiological sensor is such as a heart rhythm sensor 515 which is used to measure the heart rhythm. This heart rhythm sensor 515 is connected with a signal receiver 513, which is used to collect the physiological signals from the various sensors. The facilities at the client end may get on network 50 via the network facility 511 for transmitting the physiological data.

In the current example, the person 51 to be monitored person holds a communication set 517, for example a mobile phone. If any abnormal condition is found by the monitoring end, the communication set 517 is utilized to communicate with the remote medical staff over a voice call, video call or by texts. In one further embodiment, the positioning circuit in the communication set 517 assists the monitoring end to recognize the person's location since he/she is permitted free to walk or go out. The positioning function allows the medical staff anytime to acknowledge the person's location. It is also convenient that the monitoring end sends staff to the site if any abnormal condition is checked. Any location-based medical advice may also be provided if the person needs.

In one embodiment, the communication set at the client end is such as a smart phone or tablet computer. The program at the monitoring end is allowed to send a navigation map to the communication set at the client end according to the positioning data. Therefore, the person at the client end may accordingly find the help in accordance with the navigation map.

At the monitoring end, the servers and devices are within a local network 55. The server at the monitoring end is such as a monitoring server 551 used for retrieving physiological data from the client end. A subscriber database 553 at the monitoring end is used to store and record information of the client end. A wireless station 555 is introduced to the system for rendering wireless communication service for the monitoring terminal device 557. The device 557 is therefore allowed to wirelessly access the monitoring server 551 and the subscriber database 553.

In this architecture of telemedicine information system, the client end may be accessible to the monitoring end over the network 50 in addition to the traditional telecommunication. Over the network 50, the both sides may be conducted with voice or audio/video call if any abnormal condition is found. The mentioned notification may be incorporated to this system of notifying message over the network 50. In an exemplary embodiment, a user interface may be initiated by the program operated in the monitoring terminal device 557, and used to display the message onto the screen of the device 557. The user interface may also guide the user, e.g. the medical staff, to operate the system, including network connection, retrieval of the physiological data, and the communication made to the client end. Besides the software program installed in the communication set, a program add-on in web browser or any specific web site may serve the foregoing functionalities.

In one embodiment of the system, a message server 531 is installed. This message server 531 renders the function of transferring messages. For example, the message server 531 may render the above-mentioned service of notification, by which the messages are transferred to the corresponding client end. While the message server 531 serves as an email server, the messages are the electronic mails. While the message server 531 conducts the short message service (SMS), the message server 531 may be a simple message service server.

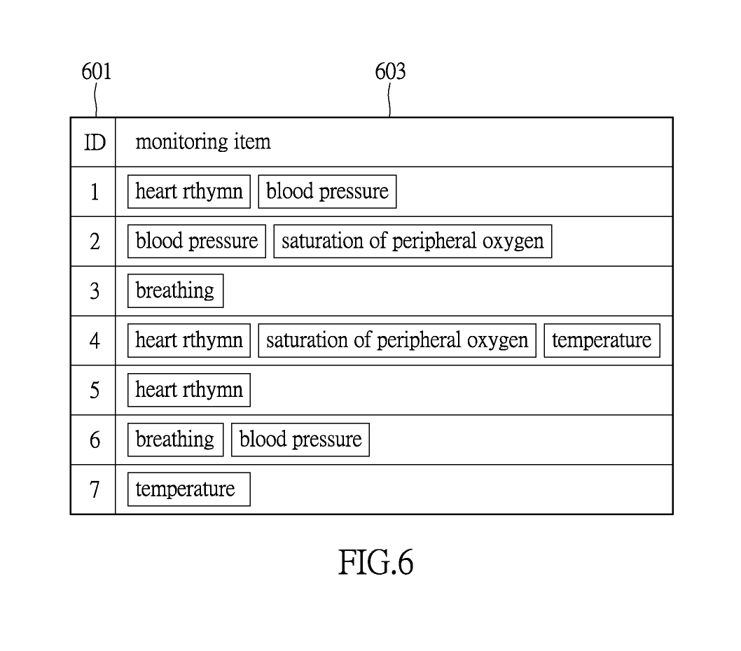

FIG. 6 schematically shows a user information screen displayed onto the monitoring terminal device.

This figure depicts the user interface of the monitoring terminal device. When the monitoring terminal device is utilized to retrieve client data associated with one or more client ends from a monitoring server, one or more monitored items 603 of the every client end may be displayed according its corresponding identification code 601. For example, the monitored items corresponding to the identification code 1 provided by the system are such as heart rhythm and blood pressure. The items corresponding to the identification code 2 are such as blood pressure and saturation of peripheral oxygen. The item for the client end with identification code 3 is such as breathing. Some other exemplary items may be clearly referred to the figure.

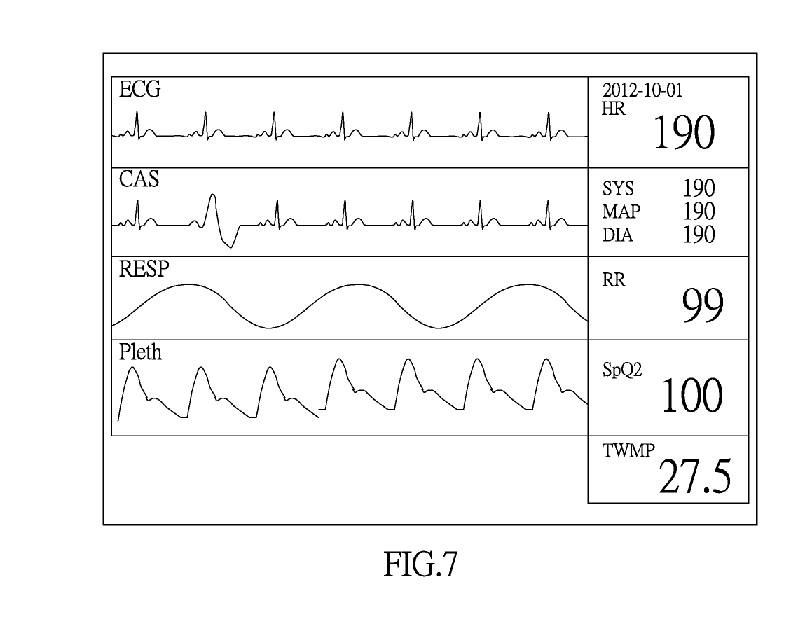

Reference is made to FIG. 7 which depicts a monitoring screen displayed on to the monitoring terminal device. One region of the user interface may be used to show the rich messages for one corresponding client end, for example an Electrocardiography (ECG) extracted from physiological data generated by an Electrocardiography machine. The result is then represented as the waves shown in the Electrocardiography. The monitoring program may provide function of alerting when any signal exceeds a specific threshold. The other applicable histogram is such as brain wave (CAS), respiratory (RESP), plethysmography (pleth) for judging cardiopulmonary condition, displaying SpO2, breathing frequency (RR), heart rate (HR), systolic blood pressure (SYS), diastolic blood pressure (DIA), and their average.

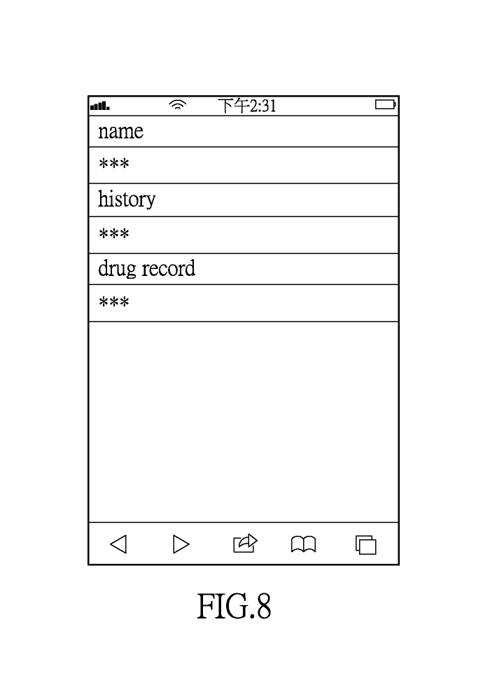

FIG. 8 further shows the client data displayed on to a computer display, and the information indicates the client's name, medical history and medication records.

To sum up the above description, the telemedicine information system in accordance with the present invention provides a terminal device held by the medical staff at a monitoring end to monitor physiological data retrieved from a client end. The terminal device may be installed with a program used to determine any condition extracted from the physiological data. Alerting message may be automatically issued if any abnormal condition is found. The medical staff may instantly make judgment and perform communication with the client for providing location-based medical advice.

The above-mentioned descriptions represent merely the preferred embodiment of the present invention, without any intention to limit the scope of the present invention thereto. Various equivalent changes, alternations or modifications based on the claims of present invention are all consequently viewed as being embraced by the scope of the present invention.

What is claimed is:

1. A telemedicine information system, comprising:
a monitoring server connected with at least one physiological sensing devices at a client end over a first network, used to acquire physiological signals generated by the at least one physiological sensing device, wherein the monitoring server is disposed with a subscriber database which records the physiological signals and associated user data;
at least one monitoring terminal device connected with the monitoring server over a second network, comprising:
a data processing unit used to process the physiological signals from the monitoring server to be monitoring information;
a communication unit, electrically connected with the data processing unit, used to establish connection over the second network to the monitoring server;
a display module, electrically connected with the data processing unit, used to display the monitoring information through a display of the monitoring terminal device;
a memory unit, electrically connected with the data processing unit, used to buffer the physiological signals retrieved from the monitoring server and the monitoring information converted from the physiological signals; and
an input module rendering an input interface and electrically connected with the data processing unit.

2. The system of claim 1, wherein the first network is a connection directed to Internet.

3. The system of claim 2, wherein every physiological sensing device at the client end establishes the connection over the first network through a communication device.

4. The system of claim 1, wherein the second network is a connection directed to a wireless local area network.

5. The system of claim 1, wherein, a mobile communication device disposed at the client end is used to receive signals transmitted from the monitoring terminal device.

6. The system of claim 5, wherein the mobile communication device is disposed with a positioning unit which is used to acquire positioning information of the mobile communication device.

7. The system of claim 5, wherein the mobile communication device is disposed with a video-audio processing unit which is used to perform audio or video call with the monitoring terminal device.

8. The system of claim 5, wherein the monitoring terminal device is a portable device which is disposed with a video-audio processing circuit which is used to perform audio or video call with the mobile communication device at the client end.

9. The system of claim 5, further comprising a message server which is forward messages between the mobile communication device at the client end and the portable device.

10. The system of claim 1, wherein the monitoring terminal device is a portable device having a touch-sensitive display.

11. The system of claim 10, further comprising a message server which is used to forward message between the mobile communication device at the client end and the portable device.
